(19) United States
(12) Patent Application Publication
(10) Pub. No.: US 2010/0048693 A1
(43) Pub. Date: Feb. 25, 2010

(54) Treatment of COPD

(75) Inventors: Mark W. Geraci, Aurora, CO (US); Patrick S. Nana-Sinkam, Columbus, OH (US)

Correspondence Address: Don D. Cha, 547 Buena Vista Road, Golden, CO, 80401, US

(73) Assignee: The Regents of the University of Colorado, A Body Corporate, Denver, CO (US)

(21) Appl. No.: 12/517,787

(22) PCT Filed: Dec. 4, 2007
(86) PCT No.: PCT/US07/86349

§ 371 Date: Jun. 4, 2009

Related U.S. Application Data
(60) Provisional application No. 60/872,985, filed on Dec. 4, 2006.

(51) Int. Cl. A61K 31/343; A61K 31/19

(52) U.S. Cl. 514/468; 514/573

(57) ABSTRACT

The present invention provides methods for treating chronic obstructive pulmonary disease. In some embodiments of the invention, methods for treating COPD comprise administering PGI₂ or a prostacyclin analog to a subject in need of such treatment.

### STATEMENT REGARDING FEDERALLY SPONSORED RESEARCH OR DEVELOPMENT

The U.S. Government has a paid-up license in this invention and the right in limited circumstances to require the patent owner to license others on reasonable terms as provided for by the terms of Grant Nos. HL077717, HL66254, HL 72340-01, and CA 58187 awarded by the National Institutes of Health.

### FIELD OF THE INVENTION

The present invention relates to methods for treating chronic obstructive pulmonary disease.

### BACKGROUND OF THE INVENTION

Chronic obstructive pulmonary disease (COPD) is characterized by a chronic inflammatory process and irreversible airflow obstruction with a decline in the lung function FEV1 (i.e., forced expiratory volume in 1 second). The disease may be divided into two subgroups, namely chronic bronchitis and emphysema. Chronic bronchitis is characterized by mucus hypersecretion from the conducting airways, inflammation and eventual scarring of the bronchi (airway tubes). Emphysema is characterized by destructive changes and enlargement of the alveoli (air sacs) within the lungs. Many persons with COPD have a component of both of these conditions. COPD patients have difficulty breathing because they develop smaller, inflamed air passageways and have partially destroyed alveoli.

The presenting symptoms for COPD are typically breathlessness accompanied by a decline in FEV1. Chronic bronchitis can also be diagnosed by asking the patient whether they have a “productive cough,” i.e. one that yields sputum. COPD patients are traditionally treated with bronchodilators and/or steroids and evaluated by spirometry for the presence of airflow obstruction and reversibility. If airflow obstruction is present and reversibility less than 15%, particularly in a smoker, then they are often diagnosed as having COPD.

At the molecular level, COPD is characterized by in increase in the activation and/or number of alveolar macrophages, CD₈⁺ T-cells and neutrophils. The neutrophil is believed to play a central role in the pathophysiology of COPD. Neutrophil activation results in the release of a number of inflammatory mediators and proteinases, most importantly neutrophil elastase which contributes to the progressive fibrosis, airway stenosis and destruction of the lung parenchyma, leading to an accelerated decline in airway function. Neutrophil elastase also induces mucus secretion and thus may contribute to the characteristic mucus hypersecretion that characterizes COPD.

About 14 million people in the United States have COPD. Currently, there is no known cure for COPD. The disease develops over many years. While COPD is caused by any pollutants or lung irritants, it is most often caused by cigarette smoking. It is believed that about 80% to 90% of COPD cases are caused by smoking, and a smoker is 10 times more likely than a nonsmoker to die of COPD. While one of the most effective methods for preventing or keeping COPD from progressing is smoking cessation, it is difficult for a smoker to quit smoking.

Accordingly there is a continuing need for COPD targeted therapies.

### SUMMARY OF THE INVENTION

One aspect of the invention provides a method for treating chronic obstructive pulmonary disease (COPD). Typically, such method generally comprises administering a therapeutically effective amount of PGI₂ or a prostacycline analog to a subject in need of such treatment. Often such method comprises administering a therapeutically effective amount of a prostacycline analog.

In some embodiments of the invention, the prostacyclin analog is selected from the group consisting of Iloprost, Beraprost, treprostenil, and a combination thereof. Within these embodiments, in some cases the prostacyclin analog is Iloprost.

In other embodiments of the invention, the method can further comprise administering to the subject a therapeutically effective amount of a bronchodilator, a corticosteroid, or a combination thereof.

Still in other embodiments of the invention, COPD is emphysema or chronic bronchitis.

While the scope of the invention includes administering the prostacyclin analog in any conventionally known manner, in some particular instances methods of the invention comprise administering of the prostacyclin analog via inhalation.

Another aspect of the invention provides a method for preventing or reducing the risk of developing COPD in a subject having a higher risk factor for developing COPD relative to a person not having a similar risk factor. Such method typically comprises administering a therapeutically effective amount of prostacyclin analog to the subject such that the risk of developing COPD in the subject is decreased by at least 10% relative to a control group with the similar risk factor in the absence of prostacyclin analog treatment.

In some embodiments, the higher risk factor for developing COPD comprises smoking an average of at least 1 pack of cigarettes per day for at least 5 years.

Yet in other embodiments, the risk of developing COPD in the subject is decreased by at least 20% relative to the control group.

Still another aspect of the invention provides a method for reducing the risk of developing pulmonary hypertension and associated morbidity in a subject having a higher risk factor for developing pulmonary hypertension relative to a person not having a similar risk factor. Such method comprises administering a therapeutically effective amount of prostacyclin analog to the subject such that the risk of developing pulmonary hypertension in the subject is decreased by at least 10% relative to a control group with the similar risk factor in the absence of prostacyclin analog treatment.

Yet another aspect of the invention provides a method for treating pulmonary hypertension of a subject. Typically such method comprises administering a therapeutically effective amount of a prostacycline analog to the subject in need of such treatment.

### BRIEF DESCRIPTION OF THE DRAWINGS

FIGS. 1A-1F show immunohistochemistry and graphic representation for prostacyclin synthase (PGI₂S) expression in human emphysema and nondiseased lung for arteriolar pulmonary endothelium and small and medium-sized vessels.

FIG. 2A is Western blotting of PGI₂S expression and 6-keto-PGF_{1α} in whole lung lysates from emphysema and nondiseased lung.

FIG. 2B is a graphic representation of the expression of PGI₂S measured by Western blotting in emphysema and nondiseased lung.

FIG. 2C is a graphic representation of PGI₂S gene expression measured by reverse transcriptase-polymerase chain reaction in emphysema and nondiseased lung.

FIGS. 3A-3D are graphic representations showing effect of cigarette smoke extract (CSE) on primary human pulmonary microvascular endothelial eicosanoid expression.

FIGS. 4A-4D show the effect of components of cigarette smoke (saturated and unsaturated aldehydes) on PGI₂S and COX-2 expression on human pulmonary microvascular endothelial cells (HPMVEC).

FIGS. 5A and 5B are graphs showing the effect of CSE exposure on HPMVEC that were pre-treated with N-acetylcysteine on PGI₂S and COX-2 expression.

FIGS. 6A and 6B are graphs showing the effect of CSE exposure on HPMVEC that were pre-treated with superoxide dismutase mimetic on PGI₂S and COX-2 expression, respectively.

FIGS. 6C and 6D are graphs showing the effect of CSE exposure on HPMVEC that were pre-treated with catalase on PGI₂S and COX-2 expression, respectively.

FIGS. 6E and 6F are graphs showing the effect of CSE exposure on HPMVEC that were pre-treated with diphenyleneiodonium chloride (DPI) on PGI₂S and COX-2 expression, respectively.

FIGS. 7A and 7B are graphs showing the effect of CSE exposure on HPMVEC that were pre-treated with N^{Ω}-nitro-l-arginine methyl ester (L-NAME) on PGI₂S and COX-2 expression, respectively.

FIGS. 8A-8C are graphs showing apoptosis in HPMVEC exposed to CSE and the effect of pre-treating HPMVEC with a prostacyclin analog Iloprost.

FIGS. 9A and 9B are immunohistochemical cleaved caspase staining of the pulmonary endothelium (arrow) of wild type and transgenic lung specific PGI₂S over-expressing mice, respectively, after six months of cigarette smoke exposure.

FIG. 9C is a graph showing quantification of cleaved caspase staining of the pulmonary endothelium in PGI₂S-overexpressing transgenic and wild-type littermates after 6 months of cigarette smoke exposure.

### DETAILED DESCRIPTION OF THE INVENTION

“Reducing the risk” in developing COPD refers to preventing or decreasing the probability of a subject developing COPD relative to a control group having a similar high risk factor but is untreated with prostacyclin analog or treated with placebo. One skilled in the art can readily determine the effectiveness of risk reduction. Such analysis typically requires a case-control study where some members of the group (case group) having a high risk of developing COPD are treated with prostacyclin analog while other members within the same group (control group) are not treated or are given placebo. To determine the effectiveness of a prostacyclin analog in reducing the incidence (or risk) of COPD development, the case-control groups are observed for a period of time that is deemed to be sufficient to provide a statistically significant analysis. As will be appreciated, the number of subjects in the case-control should be sufficient in number in order to provide a statistically significant result. In addition, animal model studies can be used to determine the effectiveness of prostacyclin analog to reduce the COPD risk.

Decrease in the risk is typically determined by comparing the differences in the incidence of COPD development between the control group and the case group after a certain period of time. Comparisons can include, but are not limited to, comparing chest radiography, pulmonary function tests (e.g., spirometry), tissue samples, cells samples, sputum samples, blood samples and the like. Any known method for comparison of these types of samples can be used to assess the relative change in the risk of developing COPD.

As used herein, a “high risk factor” refers to a factor that increases the likelihood of a subject developing COPD. Exemplary high risk factors include, but are not limited to, environmental factors, predisposed genetic factor, exposure to tobacco products, exposure to chemicals, pollutants, and other factors that are known to increase the risk of COPD. For example, smokers are at a higher risk in developing COPD compared to non-smokers. In one particular embodiment, the high risk factor refers to a subject who has been smoking at least ½ to one pack of cigarette for at least 1 year, typically at least 3 years, more typically at least 5 years, still more typically at least 10 years, and most typically at least 20 years. For the sake of brevity and clarity, the present invention will now be illustrated in reference to cigarette smoke exposure as the high risk factor; however, it should be appreciated that the scope of the invention includes other high risk factors, such as those disclosed above.

Typically, methods of the invention decrease or reduce the risk of developing COPD in the subject by at least 10% relative to a control group with the similar risk factor. Often, the risk is reduced by at least 15%, more often by at least 20%, and most often by at least 30%.

Prostacyclin analog refers to a compound, or a pharmaceutically acceptable salt thereof that acts in a similar manner as prostacyclin (prostaglandin I₂, PGI₂). In some embodiments, prostacyclin analog refers to a compound that modulates the same enzyme as PGI₂. There are a variety of in vitro assay methods available to determine whether a particular compound maybe considered as a prostacyclin analog. Any known prostacyclin analogs that are currently available can be used in methods of the invention. In one particular embodiment, the prostacyclin analog is selected from Iloprost, Beraprost, treprostenil (Remodulin), and a combination thereof. In some embodiments, methods of the invention use prostacyclin analog Iloprost.

Iloprost is a synthetic analog of prostacyclin (PGI₂) which is chemically stable and has a longer half-life than the naturally occurring substance. Iloprost is manufactured by Schering AG (Berlex Laboratories in the US). It can be administered orally, parenterally, or by inhalation. Iloprost is available for oral administration as Iloprost acid, Iloprost sodium, and Iloprost clathrate and, immediate-release tablets and capsules, and extended-release capsules.

Prostacyclin analogs can be administered in combination with other therapeutically useful agents that are conventionally used to treat COPD, such as a bronchodilator, a corticosteroid, or a combination thereof.

Unless the context requires otherwise, the term “treating” or “treatment” means (1) preventing the disease, i.e., causing the clinical symptoms of the disease not to develop in a mammal that may be exposed to or predisposed to the disease but does not yet experience or display symptoms of the disease; (2) inhibiting the disease, i.e., arresting or reducing the development of the disease or its clinical symptoms; or (3) relieving the disease, i.e., causing regression of the disease or its clinical symptoms.

“A therapeutically effective amount” means the amount of a compound that, when administered to a mammal for treating a disease, is sufficient to effect such treatment for the disease. The “therapeutically effective amount” will vary depending on the compound, the disease and its severity and the age, weight, etc., of the mammal to be treated.

Chronic obstructive pulmonary disease (COPD) can be characterized as a destruction of both small airways and parenchyma resulting in a progressive impairment in pulmonary function. Cigarette smoke is one of the major pathogenic factors implicated in COPD and pulmonary hypertension develops in approximately 6% of smokers with COPD. The interaction between parenchymal disease and the vasculature is often clinically evident by the observation that patients with severe COPD have mild or moderate pulmonary hypertension at rest. Histopathologically and microscopically, the pulmonary vasculature in COPD is typically characterized by intimal thickening with smooth muscle deposition as well as a loss of both alveolar septal structures and microvasculature. The etiology of pulmonary hypertension in COPD has been considered to be chronic hypoxemia but pulmonary hypertension is evident in milder forms of COPD where hypoxemia is absent. Accordingly, it is believed that other mechanisms maybe responsible for the vascular changes in COPD. Furthermore, in COPD it has been observed that both alveolar septal and endothelial cells undergo apoptosis. Without being bound by any theory, it is believed that this apoptosis is important to the pathogenesis of disease.

Cigarette smoke is considered to be a major risk factor in the development of COPD and its effects on the lung epithelium have been well characterized. The alveolar septae and microvasculature are both affected in COPD. As endothelial dysfunction is believed to play a role in the pathogenesis of several chronic diseases including coronary artery disease, peripheral vascular diseases, diabetes and renal failure, impaired endothelial function of pulmonary arteries has been described in small pulmonary arteries from patients with COPD. It is also believed that cigarette smoke induces necrosis and apoptosis of both epithelial and endothelial cells which contributes to the pathogenesis of COPD.

The role of eicosanoid expression in COPD and smoking related lung disease is currently not well understood. Some studies have shown elevated levels of markers of oxidative stress such as the PGF₂α analog 8-isoprostane in exhaled breath condensate (EBC) of patients with COPD. In some instances, pro-inflammatory molecules, such as leukotriene B₄ and prostaglandin E₂, have been found to be elevated in the EBC of individuals of COPD.

Without being bound by any theory, it is believed that prostacyclin (PGI₂) production occurs primarily in pulmonary vascular smooth muscle and endothelial cells via the cyclooxygenase prostaglandin H synthase pathway. Subsequent conversion to PGI₂ is believed to be mediated by prostacyclin synthase (PGI₂S), a member of the group of cytochrome P450 enzymes. It has been shown that PGI₂ has both potent vasodilatory and anti-mitogenic properties and is currently one of the main therapies for improving the survival rate of patients with severe idiopathic pulmonary arterial hypertension (IPAH). The present inventors have previously identified decreased expression of PGI₂S in the lungs from patients with severe idiopathic pulmonary arterial hypertension (IPAH). Furthermore, the present inventors have found that transgenic mice with lung specific PGI₂S over-expression were protected from hypoxia induced pulmonary hypertension.

It is believed that cigarette smoke can contribute to endothelial dysfunction and the development of cardiovascular disease through the inhibition of PGI₂ release from the endothelium. Currently, the mechanism of inhibition of PGI₂ release by cigarette smoke is not well known. Some have implicated both arachadonic acid mobilization and downstream enzymes as potential targets of cigarette smoke.

It is believed that the PGI₂ expression is reduced in other smoking related lung diseases. In addition, it is believed that reduced PGI₂ expression can be important to the pathogenesis of the disease and given the decreased survival among COPD patients with concomitant pulmonary hypertension, PGI₂ expression is believed to be protective to the pulmonary vasculature.

As stated above, COPD is believed to be predominantly related to cigarette smoke exposure. COPD is associated, among others, with varying degrees of lung function abnormalities and can lead to respiratory failure. However, it is less appreciated that major causes of morbidity and mortality in these patients are pulmonary hypertension and cardiovascular manifestations leading to coronary artery disease and stroke. Impaired endothelial cell-dependent vasodilation, inflammation, apoptosis, and proliferation are important to the endothelial dysfunction observed in smoke angiopathy and in the pathogenesis in emphysema. Endothelial cell dysfunction has been implicated in the pathogenesis of COPD

The development of COPD associated pulmonary hypertension is believed to be multifactorial and carries a significant mortality. As shown in the Examples section of this disclosure, PGI₂S expression is decreased in COPD and in HPMVEC by both cigarette smoke and acrolein. Furthermore, the Examples section also shows that PGI₂ reduces and/or prevents cigarette smoke extract (CSE) induced apoptosis of the pulmonary endothelium both in-vitro and in a murine transgenic model of PGI₂S over-expression. Without being bound by any theory, the observed decrease in lung tissue PGI₂S gene and protein expression is believed to be multifactorial and caused by oxidant stress, loss of alveolar capillary endothelial cells, nitric oxide-related suppression of the PGI₂S protein release and altered transcriptional control.

Furthermore, it is believed that PGI₂ expression is biologically relevant to the pulmonary vasculature in smoking related lung disease including COPD. Accordingly, one aspect of the invention provides a method for treating, preventing or reducing the symptoms of COPD by administering a PGI₂ or a prostacyclin analog to the subject in need of such a treatment.

As shown in the examples section, CSE and acrolein reduced the expression of the PGI₂S gene and CSE increased the expression upstream mediators of the eicosanoid pathway (COX-2 and cPLA2) in HPMVEC. Cigarette smoke contains over 4,000 compounds including acrolein, which is believed to play a role in cigarette smoke induced lung toxicity and potentially in cigarette smoke induced lung cancer. It is believed that acrolein mediates pulmonary inflammation through the induction of inflammatory cytokines and inhibition of neutrophil apoptosis. These biological changes are believed to contribute to COPD pathogenesis. In addition, acrolein is also believed to contribute to endothelial dysfunction through the depletion of glutathione and subsequent oxidative stress. It should be noted that although CSE provides severe oxidative stress to the cultured endothelial cells, a number of antioxidant pretreatment strategies did not prevent CSE-induced decrease in PGI₂S gene expression.

Accordingly, another aspect of the invention provides a method for treating pulmonary inflammation. In one embodiment, pulmonary inflammation is treated by administering a compound that is capable of reducing or inhibiting induction of inflammatory cytokines caused by a high risk factor, such as cigarette smoke or exposure thereto. In another embodiment, pulmonary inflammation is treated by administering a compound that is capable of reducing and/or preventing inhibition of neutrophil apoptosis by a high risk factor, such as cigarette smoke.

Still another aspect of the invention provides a method for treating COPD by inducing PGI₂S gene expression. In some embodiments, a compound that is capable of inducing PGI₂S gene expression in lung endothelial cells is administered to a subject in need of such treatment. Whether a particular compound can induce PGI₂S gene expression can be readily determined by one skilled in the art by any of the various techniques available, such as by measuring the amount of PGI₂S gene expression in vitro or in vivo assay after administering the compound. Some such techniques are disclosed in the Examples section of this disclosure.

As described in the Examples section, it has been shown that CSE differentially and in a substantially dose-dependent manner affects PGI₂S gene expression and causes apoptotic cell death in HPMVEC. Without being bound by any theory, whereas CSE induced a degree of endothelial cell apoptosis, it is believed that the decrease in PGI₂S mRNA is unlikely the result of apoptotic cell loss since the gene expression of both upstream enzymes cPLA₂ and COX-2 increased in these cells. Furthermore, the present inventors have shown that pretreatment with the prostacyclin analog (e.g., Iloprost) conferred at least partial protective anti-apoptotic effect.

It is believed that cigarette smoke induces oxidative stress. The imbalance in eicosanoid gene expression is believed to be, in some instances, mediated by oxidative stress. In addition, as shown in the Examples section, it appears that acrolein is one of the key components of cigarette smoke responsible for PGI₂S suppression. Accordingly, another aspect of the invention provides a method for treating COPD by administering a compound that is capable of reducing the PGI₂S suppression effect of acrolein in lung endothelial cells, e.g., a compound that is acrolein antagonist.

As shown herein, CSE and acrolein appear to have direct suppressive effects on PGI₂S gene expression rather than upstream mediators. There are several potential mechanisms for PGI₂S suppression including, but not limited to, transcriptional regulation by methylation, promoter base pair rearrangement by oxidative stress and alteration in transcriptional binding factors. The observation that acrolein decreased PGI₂S expression while several other aldehydes did not appears to suggest a mechanism specific to acrolein. Without being bound by any theory, it is believed that in some instances acrolein interferes with transcriptional regulation of genes and preferentially binds to CpG sites. As shown herein, it also appears that an imbalance in eicosanoid expression may be relevant to the observed vascular toxicity of acrolein.

Chronic obstructive lung disease is associated with varying degrees of lung function abnormalities and can lead to respiratory failure. However, it is less appreciated that major causes of morbidity and mortality in these patients are pulmonary hypertension and cardiovascular manifestations leading to coronary artery disease and stroke. Impaired endothelial cell dependent vasodilation, inflammation, apoptosis and proliferation are believed to be significant to the endothelial dysfunction observed in smoke angiopathy and/or the pathogenesis in COPD.

As shown herein, reduced PGI₂S expression in the pulmonary endothelium of long-standing smokers is at least one of the factors associated with emphysema. Moreover, the present inventors have shown that PGI₂S expression is both diminished in the pulmonary endothelium in COPD and that PGI₂ confers anti-apoptotic effects to the pulmonary endothelium following both acute and chronic cigarette smoke exposure.

Accordingly, another aspect of the invention provides a method for treating COPD by administering PGI₂ or its analog. In some embodiments, methods of the invention comprise treating a subject with a compound that is capable of conferring anti-apoptotic effects to the pulmonary endothelium cells.

### Formulations

Prostacyclin analogs can be administered to a subject to achieve a desired physiological effect. Typically the subject is an animal, more typically a mammal, and most often a human. The Prostacyclin analog can be administered in a variety of forms adapted to the chosen route of administration, e.g., orally or parenterally. Parenteral administration in this respect includes administration by the following routes: intravenous; intramuscular; subcutaneous; intraocular; intrasynovial; transepithelially including transdermal, ophthalmic, sublingual and buccal; topically including ophthalmic, dermal, ocular, rectal and nasal inhalation via insufflation and aerosol; intraperitoneal; and rectal systemic.

The prostacyclin analog can be orally administered including via inhalation, for example, with an inert diluent or with an assimilable edible carrier, or it can be enclosed in hard or soft shell gelatin capsules, or it can be compressed into tablets, or it can be incorporated directly with the food of the diet. For oral therapeutic administration, the prostacyclin analog may be incorporated with excipient and used in the form of ingestible tablets, buccal tablets, troches, capsules, elixirs, suspensions, syrups, wafers, and the like. Such compositions and preparation can contain at least 0.1% of active prostacyclin analog. The percentage of the compositions and preparation can, of course, be varied and can conveniently be between about 1 to about 10% of the weight of the unit. The amount of active prostacyclin analog in such therapeutically useful compositions is such that a suitable dosage will be obtained. Typically compositions or preparations according to the present invention are prepared such that an oral dosage unit form contains from about 1 to about 1000 mg of active prostacyclin analog. In one particular embodiment, prostacyclin analog is administered orally or by an aerosol delivery system.

The tablets, troches, pills, capsules and the like can also contain the following: a binder such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, lactose or saccharin can be added or a flavoring agent such as peppermint, oil of wintergreen, or cherry flavoring. When the dosage unit form is a capsule, it can contain, in addition to materials of the above type, a liquid carrier. Various other materials can be present as coatings or to otherwise modify the physical form of the dosage unit. For instance, tablets, pills, or capsules can be coated with shellac, sugar or both. A syrup or elixir can contain the prostacyclin analog, sucrose as a sweetening agent, methyl and propylparabens a preservatives, a dye and flavoring such as cherry or orange flavor. Of course, any material used in preparing any dosage unit form should be pharmaceutically pure and substantially non-toxic in the amounts employed. In addition, the prostacyclin analog can be incorporated into sustained-release preparations and formulation.

The prostacyclin analog can also be administered parenterally. Solutions of the prostacyclin analog can be prepared in water suitably mixed with a surfactant such as hydroxypropylcellulose. Dispersion can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It can be stable under the conditions of manufacture and storage and must be preserved against the contaminating action of microorganisms such as bacterial and fungi. The carrier can be a solvent of dispersion medium containing, for example, water, ethanol, polyol (e.g., glycerol, propylene glycol, and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, e.g., sugars or sodium chloride. Prolonged absorption of the injectable compositions of agents delaying absorption, e.g., aluminum monostearate and gelatin.

Sterile injectable solutions are prepared by incorporating the prostacyclin analog in the required amount in the appropriate solvent with various other ingredients enumerated above, as required, followed by filtered sterilization. Generally, dispersions are prepared by incorporating the various sterilized active ingredient into a sterile vehicle which contains the basic dispersion medium and the required other ingredients from those enumerated above. In the case of sterile powders for the preparation of sterile injectable solutions, the preferred methods of preparation are vacuum drying and the freeze drying technique which yield a powder of the active ingredient plus any additional desired ingredient from previously sterile-filtered solution thereof.

The prostacyclin analog can be administered alone or in combination with pharmaceutically acceptable carriers, as noted above, the proportion of which is determined by the solubility and chemical nature of the prostacyclin analog, chosen route of administration and standard pharmaceutical practice.

The physician will determine the dosage of the prostacyclin analog which will be most suitable for prophylaxis or treatment and it will vary with the form of administration and the particular prostacyclin analog chosen, and also, it will vary with the particular patient under treatment. The physician will generally wish to initiate treatment with small dosages by small increments until the optimum effect under the circumstances is reached. The therapeutic dosage can generally be from about 0.1 to about 1000 mg/day, and preferably from about 10 to about 100 mg/day, or from about 0.1 to about 50 mg/Kg of body weight per day and preferably from about 0.1 to about 20 mg/Kg of body weight per day and can be administered in several different dosage units. Higher dosages, on the order of about 2× to about 4×, may be required for oral administration.

Additional objects, advantages, and novel features of this invention will become apparent to those skilled in the art upon examination of the following examples thereof, which are not intended to be limiting. Procedures that are constructively reduced to practice are described in the present tense, and procedures that have been carried out in the laboratory are set forth in the past tense.

### EXAMPLES

### Methods

### Cell Culture and Materials

Human pulmonary microvascular endothelial cells (HPMVEC, passage 4-6) and EGM-2-MV medium (containing 5% FBS, hydrocortisone, human recombinant VEGF, recombinant human fibroblast growth factor-B, recombinant insulin-like growth factor-1, human recombinant epidermal growth factor, ascorbic acid, gentamycin and amphotericin-B) were purchased from Clonetics Corporation (Baltimore, Md., USA). Cells were grown to confluency at 37° C. in a humidified atmosphere of 21% O₂, 5% CO₂. N-acetyl-L-cysteine (NAC) and diphenyleneiodonium chloride (DPI) were obtained from Sigma-Aldrich (St. Louis, Mo., USA). N-nitro-l-arginine methyl ester (L-NAME) was obtained from Cayman Chemical (Ann Arbor, Mich.). Z-Asp2,6-dichlorobenzoylmethylketone (Caspase Family Inhibitor IV) was obtained from Alexis Biochemicals (San Diego, Calif., USA). Both an SOD mimetic and catalase were obtained from National Jewish Medical Center. Acrolein, an α,β-unsaturated aldehyde, and several saturated aldehydes (acetaldehyde, crotonaldehyde, proprionaldehyde (i.e., propanal) were purchased from Sigma Aldrich (St. Louis, Mo., USA)

### Preparation of Cigarette Smoke Extract (CSE)

Cigarette smoke extract was prepared by a modification of a previously published method, e.g., Carp et al., Am. Rev. Respir. Dis., 1978, 118, 617-621. Briefly, one non-filtered Camel cigarette (R.J. Reynolds, Winston-Salem, N.C.) was passed through 10 mL of Phosphate-buffered saline (PBS) using a vacuum pump. This 100% CSE was adjusted to pH 7.4 and filtered through a 0.22 μM pore filter (Fisher, Hampton, N.H., USA) and the CSE was diluted to the appropriate concentration and added to endothelial cells within 10 min of preparation.

### Immunohistochemistry

Paraffin embedded sections of human lung tissue (obtained from the University of Colorado Tissue bank, four emphysema and four non-diseased) were deparaffinized and rehydrated with xylene and ethanol. Antigen retrieval was performed using the microwave method with citrate buffer for 20 min. Avidine and biotin block was performed and endogenous peroxidase was quenched by 3% hydrogen peroxide. After blocking with 5% normal goat serum, rabbit anti-human PGI₂S antibody (1:25) was applied overnight at 4° C. The sections were washed with PBS with 0.05% Tween and incubated with biotinylated goat anti-rabbit IgG. After washing, sections were stained with ABC Vectastatin reagents (Vector Labs, Burlingame, Calif.; PK-6101) and DAB (Vector Labs. SK4100). Count staining was done by hematoxylin dye and sections were hydrated and mounted for microscopy.

### Apoptosis Assay

Immunohistochemical staining on both PGI₂S transgenic mice and wild type littermates was conducted using similar protocols to the above, however, cleaved caspase 3 antibody (Cell Signaling technology Danvers, Mass.) was used to assess apoptosis. In a blinded fashion, small, medium and large pulmonary arteries were scored for endothelial caspase staining. In-vitro, the rate of apoptosis in primary human microvascular endothelial cells was analyzed by the Vybrant Apoptosis Assay Kit from Molecular Probes Inc. (Eugene, Oreg., USA) according to the manufacturer's protocol. After treatment with CSE, the cells were washed and trypsinized. 1×10⁶ of cells were suspended in 100 μL of Annexin-binding buffer and FITC annexin-V/propidium iodide (PI) working solution was added. After 15 minutes of incubation, samples were analyzed by flow cytometry measuring the fluorescence emission at 530 nm (e.g., FL1) and >575 nm (e.g., FL3). In addition, apoptosis of HPMVEC was assessed by Caspase 3/7 assay according to the protocol provided (Promega Madison, Wis.).

### Western Blotting

20 μg of protein was separated on NuPAGE Novex 4-12% Bis-Tris Gel (Invitrogen Carlsbad, Calif., USA) and transferred to a polyvinylidene fluoride (PVDF) membrane (Invitrogen, Carlsbad, Calif., USA). The membrane was blocked with 5% nonfat dry milk in PBS containing 0.05% Tween (PBST) for 1 hour. After washing with PBST, the membrane was incubated with rabbit polyclonal PGI₂S antibody (1:1,000) overnight at 4° C. The membrane was washed 4 times and incubated with horseradish peroxidase (HRP) conjugated secondary antibody (anti-rabbit, 1:3,000) (Santa Cruz, Santa Cruz, Calif.) for 1 hour. Film was developed by chemiluminescence (Perkin Elmer, Boston, Mass.).

### Real Time RT-PCR

Total RNA was extracted using the RNeasy Mini Kit (Qiagen, Valencia, Calif.), following the manufacturer's instructions. 1 μg of RNA was reverse-transcribed using random primer and MultiScribe RT (High-Capacity cDNA Archive Kit, Applied Biosystems, Foster City, Calif.). Assay-on-demand gene expression probe for PGI₂S: Hs00168766, COX-1:Hs00377721, COX-2:Hs00153133, cPLA₂:Hs00233352, and β-actin: Hs999999903 were obtained from Applied Biosystems (Foster City, Calif.) PCR reactions were performed in 20 μL volumes containing 9 μL of cDNA, 10 μL of TaqMan Master Mix (Applied Biosystems) and 1 μL of assay-on-demand primer and probe. Real-time PCR was conducted on the Applied Biosystems GeneAmp 5700 sequence Detection System and signal was detected by the GeneAmp 5700 SDS software (Applied Biosystems). All values were reported relative to β-Actin expression.

6-keto-PGF1 Measurements

6-keto-PGF_{1α} was measured by competitive ELISA. Endothelial cell supernatants, antibody (mouse anti-PGE or rabbit anti-6KETO PGF_{1α}), and tracer were added to wells on plates coated with anti-rabbit (6-keto-PGF_{1α}) antibodies. The tracer is 6-keto-PGF_{1α} linked to acetylcholinesterase. This mixture was left overnight at 4° C. The plates were then washed, and Ellman's reagent was added (acetylthiocholine iodide and 5,5′-dithiobis-(2-nitrobenzoic acid) in a 1 M phosphate buffer). The samples were read in a spectrophotometer at 405 nm. Antibodies and tracer were obtained from Cayman Chemicals (Ann Arbor Mich.).

### Murine Model of PGI₂S Over-expression

As described previously, transgenic mice were developed using a construct consisting of the human SP-C promoter and full-length rat prostacyclin synthase cDNA. See, for example, Keith et al., Cancer Res., 2004, 64, 5897-5904. The SP-C promoter allows targeted expression to alveolar and distal airway epithelial cells. Genotyping of animals was conducted by performing PCR on genomic DNA isolated from tails. Each line was propagated as heterozygotes. Transgenic mice (Tg⁺) were always bred with wild-type FVB/N (Jackson Labs, Bar Harbor, Me.) mice to produce the experimental Tg⁺ mice and transgenic negative littermates (Tg⁻), which were used as controls in all of the experiments. Transgenic (n=3) and wild type (n=3) mice were exposed to 22 weeks of mainstream cigarette smoke.

### Statistical Analysis

All statistical analysis was conducted in the initial studies in human lung tissue by T-test (P<0.05). All in vitro studies were conducted by one way analysis of variance with statistical significance at P<0.05.

### Results

PGI₂S Protein Expression in Lung Tissue from Patients with Emphysema and Normal Lung

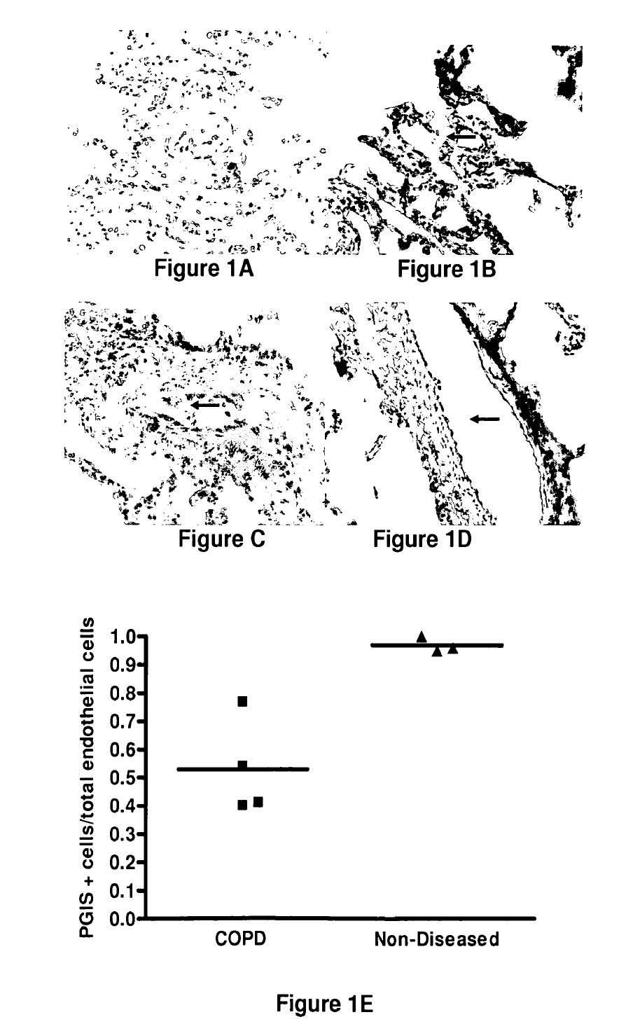

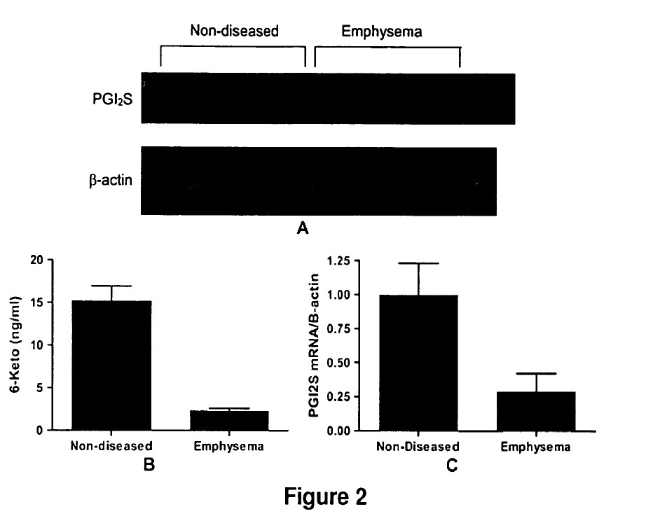

Immunohistochemistry was performed for PGI₂S on human lung tissue from four individuals with emphysema and three individuals with normal lung parenchyma. Scoring of endothelial cells for PGI₂S expression as a factor of total endothelial cells revealed that PGI₂S was decreased primarily in the endothelial cell monolayer of arterioles in the lungs from patients with pulmonary emphysema (FIG. 1A) compared with normal subjects (FIGS. 1B and 1E). PGI₂S staining in medium-sized pulmonary vessels in emphysema and nondiseased lung was comparable (FIGS. 1C, 1D and 1F) Western analysis of whole-lung lysates for PGI₂S (FIG. 2A) confirmed that protein expression was decreased (by ˜50% by densitometry) in lung tissue extracts from the emphysema lungs compared with normal control lung tissue extracts.

Referring again to FIG. 1, there was a reduction in PGI₂S staining (arrow) of arteriolar pulmonary endothelium from a patient with emphysema (FIG. 1A) compared with nondiseased lung (FIG. 1B). In small and medium-sized vessels, staining was similar in the endothelium from a patient with emphysema (FIG. 1C) compared with nondiseased lung (FIG. 1D) (arrow) (original magnification: ×40). Sections were scored by a pathologist in blinded fashion (number of PGI₂S positive endothelial cells/100 endothelial cells per case) for PGI₂S staining and expressed as a ratio in capillaries, small/medium arteries, and arterioles. The differences in PGI₂S staining were statistically significant in pulmonary arteriolar endothelium (FIG. 1E) (0.53±0.086 vs. 0.97±0.015; p±0.01) but not in small/medium-sized vessels (FIG. 1F) (0.74±0.12 vs. 0.97±0.005; p±0.16).

Western analysis for PGI₂S (FIG. 2A) confirmed that protein expression was decreased (approximately by 50% by densitometry) in lung tissue extracts from the emphysema lungs when compared to normal control lung tissue extracts. As can be seen in FIG. 2, the expression of PGI₂S measured by Western blotting was decreased in patients with COPD (FIG. 2A).

6-keto-PGF_{1α} (the Stable Metabolite of PGI₂ ) in Emphysema Lungs

To examine whether the reduced amount of lung tissue PGI₂S protein was associated with decreased amounts of PGI₂S, the stable metabolite of PGI₂ (6-keto-PGF_{1α}) was measured by ELISA. As shown in FIG. 2A, the expression of PGI₂S measured by Western blotting is decreased in patients with emphysema. The amount of 6-keto-PGF_{1α} measured per mg of lung tissue homogenate was significantly reduced in lung tissue samples from patients with emphysema by about 75% (p<0.05). See FIG. 2B. Lung tissue samples were examined using real time PCR, which showed decreased PGI₂S mRNA expression in the lungs from emphysema patients when compared to normal lung tissue (p<0.05). See FIG. 2C.

### Effect of CSE on Eicosanoid Gene Expression in HPMVEC

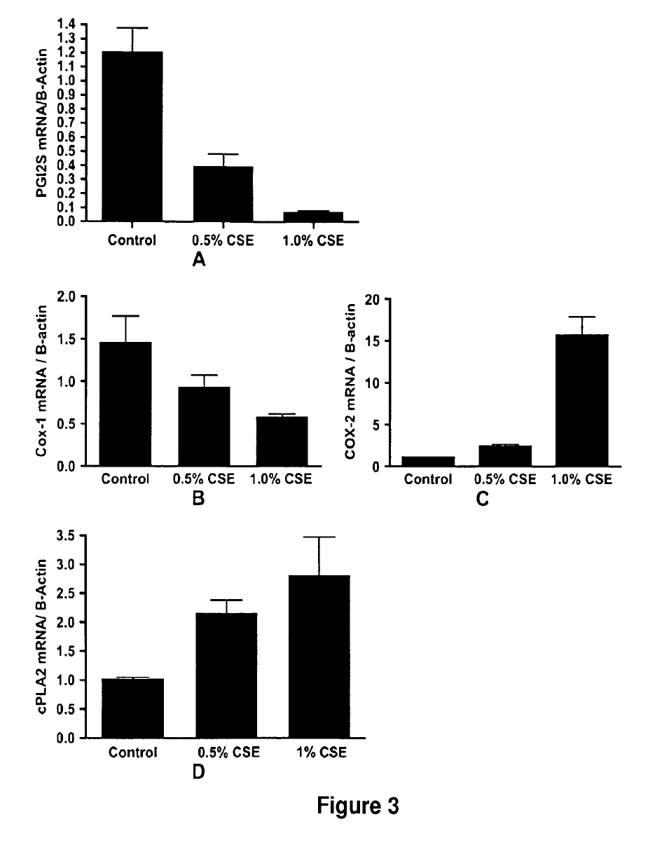

Primary human pulmonary microvascular endothelial cells (HPMVEC between 4th to 6th passages) were treated with CSE (0.5% and 1%) and then examined and different time points for gene expression (12 hours, 24 hours and 48 hours). As can be seen, PGI₂S gene expression was decreased in a substantially dose dependent manner with a maximum effect at about 24 hours after incubation with CSE (p<0.05). See FIG. 3A. For the remainder of the experiments, endothelial cells were treated with 0.5% and 1% CSE and the gene expression was examined at 24 hours. COX-1 gene expression was decreased (see FIG. 3B) by 1% CSE (P<0.05) while COX-2 and cytosolic phospholipase A₂ (cPLA₂) were significantly increased (about 10 fold and about three fold, respectively) (P<0.05). See FIGS. 3C and 3D.

### Effect of Aldehydes on Eicosanoid Gene Expression

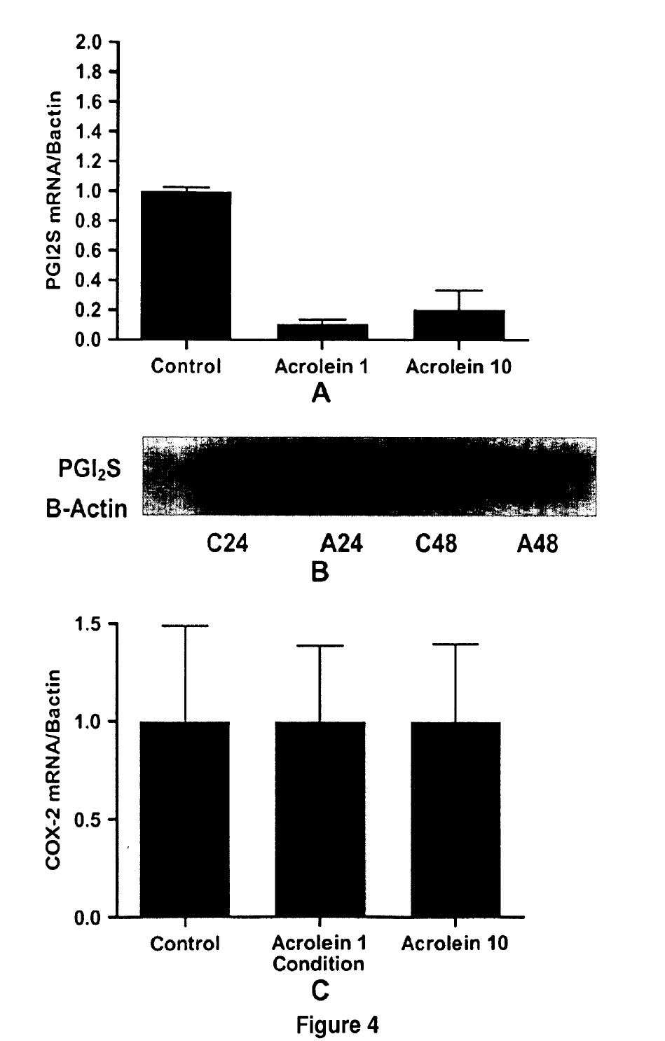

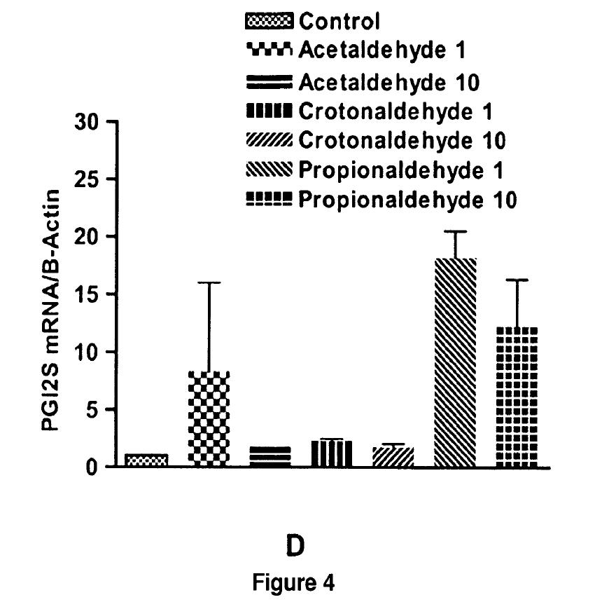

HPMVEC were exposed to 1 and 10 μM of unsaturated aldehydes (acrolein) and saturated aldehydes (acetaldehyde, crotonaldehyde, proprionaldehyde) and assessed for eicosanoid expression. Acrolein caused a reduction in PGI₂S gene expression about 24 hours after exposure at both concentrations and a trend toward induction in COX-2 gene expression. See FIG. 4A. In addition, acrolein suppressed PGI₂S protein expression at about 48 hours post exposure. See FIG. 4B. No significant change in COX-2 gene expression was noted after acrolein exposure (FIG. 4C). Administration of other aldehydes (acetaldehyde, crotonaldehyde, and propionaldehyde) did not result in suppression of PGI₂S mRNA expression. Propionaldehyde resulted in a statistically significant induction in PGI₂S expression (FIG. 4D) (p<0.001). All studies were conducted in triplicate on separate days. A=acrolein; C=control

Effects of Pretreatment with Anti-oxidant Agents on CSE-induced Gene Expression Changes in Eicosanoid Pathway Enzymes

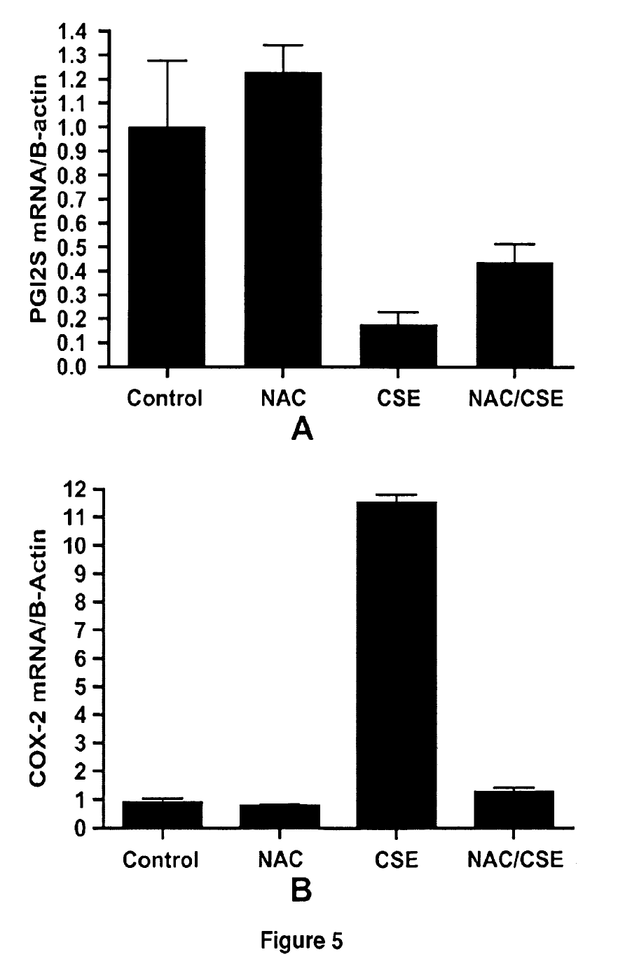

Whether experimental antioxidant strategies would protect against the CSE-induced decrease of PGI₂S expression in human pulmonary microvascular cells was examined. HPMVEC were pre-incubated for four hour with N-Acetyl-L-cysteine (NAC) (1 mM) and then exposed to 1% CSE. The cells were then examined via RT-PCR at four hours and 24 hours, which showed no significant prevention of down-regulation of PGI₂S gene expression by 1% CSE (see FIG. 5A), but the data showed that NAC did significantly reduce or prevent the CSE mediated induction of COX-2 gene expression (P<0.05) (see FIG. 5B).

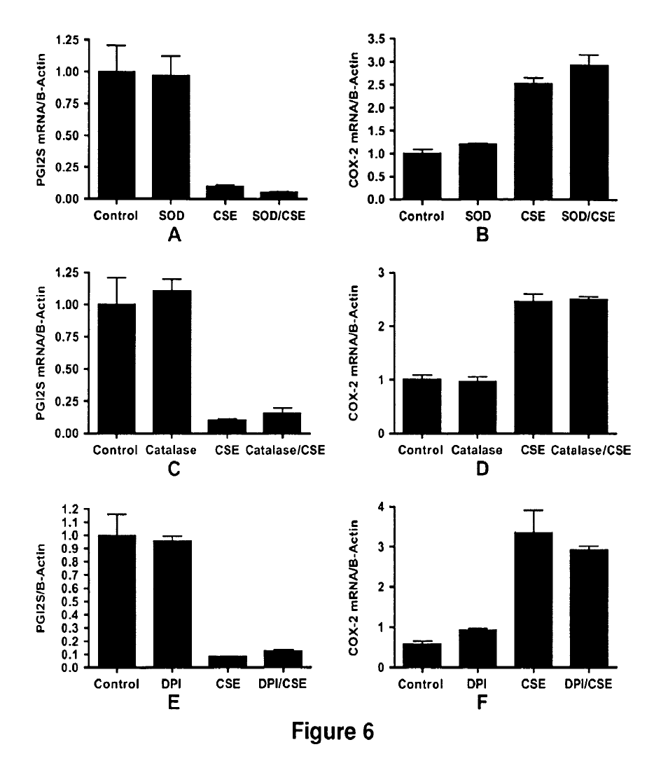

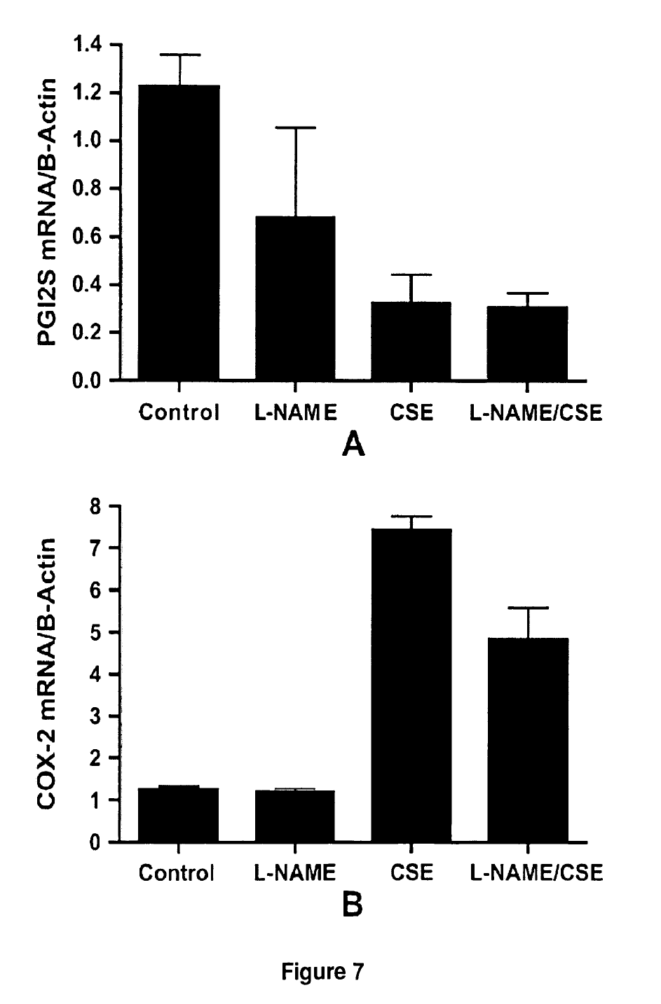

Four hour pre-incubation of endothelial cells with a superoxide dismutase (SOD) mimetic (100 U/ml) (FIGS. 6A and 6B), catalase (100 U/ml) (FIGS. 6C and 6D), diphenyleneiodon-ium chloride (DPI), a nitric oxide inhibitor, (0.1 μM) (FIGS. 6E and 6F) or L-NAME (NOS inhibitor) (1 mM) (FIG. 7A) did not significantly affect the PGI₂S gene expression and COX-2 gene expression when the cells were exposed to 1% CSE for 4 hours. There was a trend toward the reversal of induction of COX-2 expression in cells treated with L-NAME (FIG. 7B).

### Effect of CSE and Iloprost on HPMVEC Apoptosis

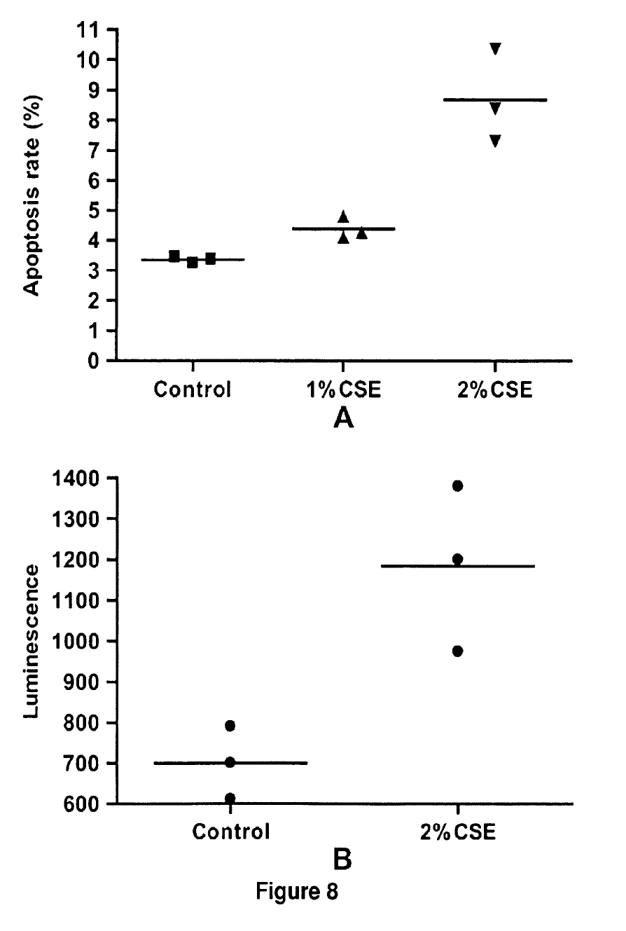

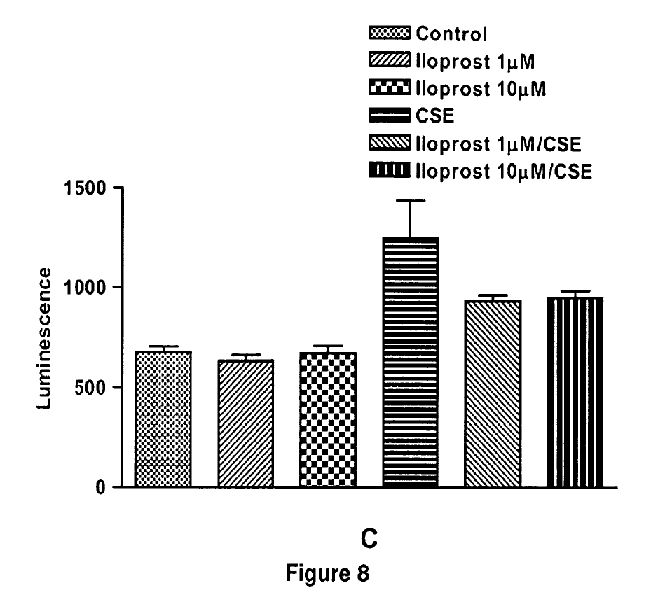

Incubation of HPMVEC with 1% and 2% CSE increased the apoptosis rate at 24 hours from 3% in control cells to 9% (as measured by flow cytometry for fluorescence) in cells treated with 2% CSE. See FIGS. 8A and 8B (control versus 2% CSE post 24 hours) (P<0.05). Induction of apoptosis was confirmed by both annexin V and caspase 3/7 analysis. FIG. 8B.

When the cells were pre-treated with a prostacyclin analog Iloprost (1 μM and 10 μM) for 30 minutes prior to CSE exposure, a significant reduction in endothelial apoptosis, as measured by caspase 3/7 activity, was observed. FIG. 8C.

### Effect on Chronic CSE Exposure in PGI2S Transgenic Animals

The present inventors have shown that transgenic mice with lung specific over-expression of PGI₂S were partially protected from both chemical induced and cigarette smoke induced lung tumors. See Keith et al., Cancer Res., 2004, 64, 5897-5904.

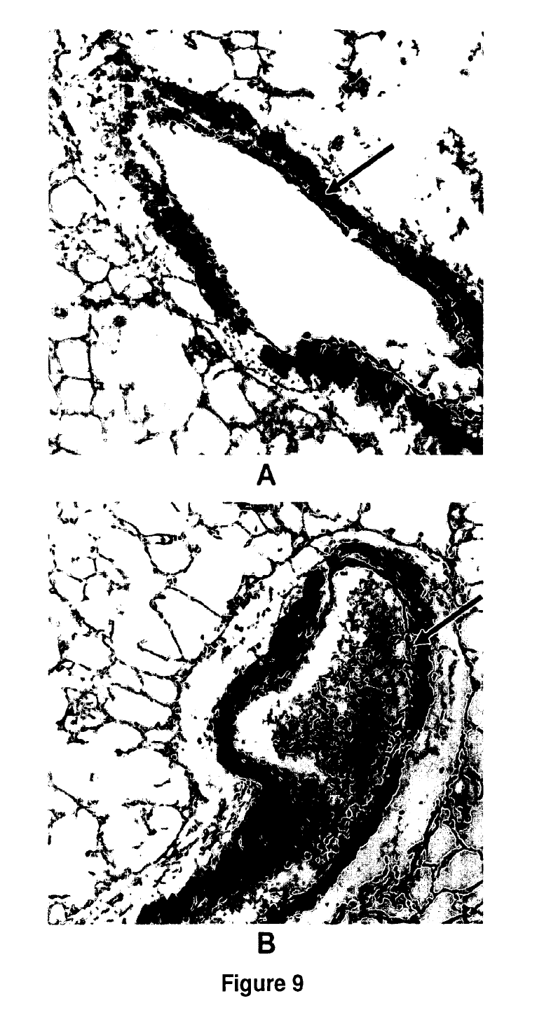

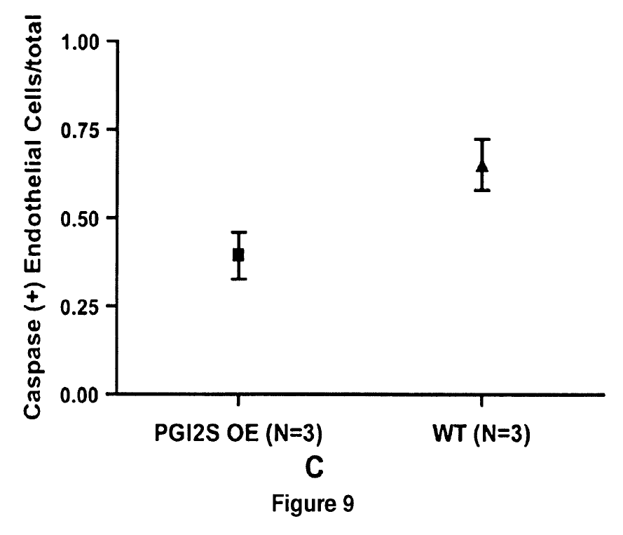

Wild-type (FIG. 9A) and PGI₂S transgenic mice (FIG. 9B) (FVB background) were exposed to 6 months of cigarette smoke. Immunohistochemistry and subsequent scoring for cleaved caspase activity per total endothelial cells revealed a significant reduction in caspase 3 expression in transgenic mice (0.3946±0.06583) compared with wild-type littermates (0.6521±0.07224), suggesting decreased apoptosis. FIG. 9C.

FIGS. 9A and 9B show immunohistochemical cleaved caspase staining of the pulmonary endothelium (arrow) of wild type and transgenic lung specific PGI₂S over-expressing mice, respectively, after 6 months of cigarette smoke exposure (original magnification: ×200). Results represent the number of caspase-positive endothelial cells per total number of endothelial cells as a ratio. Quantification of cleaved caspase staining of the pulmonary endothelium in PGI₂S-overexpressing transgenic (PGI₂S OE) (0.3946±0.06583) was significantly decreased in comparison to wild-type (WT) (0.6521±0.07224) littermates after 6 months of cigarette smoke exposure. FIG. 9C. Vessel scoring was conducted in blinded fashion in six vessels per mouse (total, 18 vessels) in each experimental group (p<0.05). OE=over expressor

The foregoing discussion of the invention has been presented for purposes of illustration and description. The foregoing is not intended to limit the invention to the form or forms disclosed herein. Although the description of the invention has included description of one or more embodiments and certain variations and modifications, other variations and modifications are within the scope of the invention, e.g., as may be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. A method for treating chronic obstructive pulmonary disease (COPD) comprising administering a therapeutically effective amount of a prostacycline analog to a subject in need of such treatment.

2. The method of claim 1, wherein the prostacyclin analog is selected from the group consisting of Iloprost, Beraprost, treprostenil, and a combination thereof.

3. The method of claim 1, wherein the subject is also administered with a therapeutically effective amount of a bronchodilator, a corticosteroid, or a combination thereof.

4. The method of claim 1, wherein COPD is emphysema or chronic bronchitis.

5. The method of claim 1, wherein the prostacyclin analog is administered via inhalation.

6. A method for reducing the risk of developing COPD in a subject having a higher risk factor for developing COPD relative to a person not having a similar risk factor, said method comprising administering a therapeutically effective amount of prostacyclin analog to the subject such that the risk of developing COPD in the subject is decreased by at least 10% relative to a control group with the similar risk factor in the absence of prostacyclin analog treatment.

7. The method of claim 6, wherein COPD is emphysema or chronic bronchitis.

8. The method of claim 6, wherein the higher risk factor for developing COPD comprises smoking an average of at least 1 pack of cigarettes per day for at least 5 years.

9. The method of claim 6, wherein the prostacyclin analog is selected from the group consisting of Iloprost, Beraprost, treprostenil, and a combination thereof.

10. The method of claim 6, wherein the risk of developing COPD in the subject is decreased by at least 20% relative to the control group.

11. A method for reducing the risk of developing pulmonary hypertension in a subject having a higher risk factor for developing pulmonary hypertension relative to a person not having a similar risk factor, said method comprising administering a therapeutically effective amount of prostacyclin analog to the subject such that the risk of developing pulmonary hypertension in the subject is decreased by at least 10% relative to a control group with the similar risk factor in the absence of prostacyclin analog treatment.

12. The method of claim 11, wherein the prostacyclin analog is selected from the group consisting of Iloprost, Beraprost, treprostenil, and a combination thereof.

13. The method of claim 11, wherein the risk of developing pulmonary hypertension in the subject is decreased by at least 20% relative to the control group.

14. A method for treating pulmonary hypertension of a subject, said method comprising administering a therapeutically effective amount of a prostacycline analog to the subject in need of such treatment.

15. The method of claim 14, wherein the prostacyclin analog is selected from the group consisting of Iloprost, Beraprost, treprostenil, and a combination thereof.

16. The method of claim 14, wherein the subject is also administered with a therapeutically effective amount of a bronchodilator, a corticosteroid, or a combination thereof.
